(19) United States
(12) Patent Application Publication
(10) Pub. No.: US 2006/0248660 A1
(43) Pub. Date: Nov. 9, 2006

(54) Hair product packaging and methods

(76) Inventors: Steven P. Ryan, Omaha, NE (US); David G. Wood, Omaha, NE (US); Don Ryan, Beaumont, TX (US); Wayne L. Ryan, Omaha, NE (US)

Correspondence Address: DOBRUSIN & THENNISCH PC, 29 W LAWRENCE ST, SUITE 210, PONTIAC, MI, 48326, US

(21) Appl. No.: 11/120,329

(22) Filed: May 3, 2005

(51) Int. Cl. A61K 8/00

(52) U.S. Cl. 8/405

(57) ABSTRACT

A method for delivering hair coloring products, including the steps of filling a package with a granulated oxidizer and transporting the resulting filled package to a remote site (such as a hair salon) for mixing with a liquid, a hair colorant or a combination thereof. Also contemplated is an improved solid-state mixture adapted for point-of-use hair coloring product formation.

### TECHNICAL FIELD

The present invention relates generally to hair coloring products, and more particularly to improvements in packaging of ingredients of hair coloring systems that employ a concentrated (e.g., granulated) oxidizer, such as urea peroxide powder.

### BACKGROUND

It is known to use liquid peroxide solutions alone or in combination with a hair colorant for changing the appearance of hair. Wide varieties of products currently exist, including but not limited to retail products sold for home use by consumers and products sold directly to beauty salons for use by or under the supervision of professional cosmetologists.

Handling of liquid peroxide solutions generally requires appropriate care to avoid spillages and contact with skin, clothing and other articles. It is also recognized that, particularly for bulk quantities of liquid peroxide solutions required for large scale use in salons, there is a substantial cost associated with packaging and transport of the solutions. The nature of the ingredients, the weight of the liquid and the space occupied by the required volumes generally make such packaging and transport inconvenient. In addition, once the solutions arrive at a salon, or retail consumers purchase retail products, the resulting end users need to take appropriate care in their own handling and storage of the products.

Another issue faced by users of liquid peroxides is the tendency for peroxide concentration to change during storage, owing to its intrinsic stability characteristics.

What has become a recognized need in the field, therefore, is an approach to hair coloring products that avoids the inconveniences posed by current commercial approaches.

### SUMMARY OF THE INVENTION

The present invention meets one or more of the above needs by providing an improved approach for the manufacture and delivery of hair coloring products to an end user, as well as improved hair coloring products. More specifically, in one aspect (though other variations will be apparent from the discussion that follows), the invention is directed to a method for delivering hair coloring products, comprising the steps of causing a package that is substantially free of liquid to be filled to at least a first predetermined amount (e.g., at least 0.1 kilogram) with a concentrate of an oxidizer, such as a granulated mixture including an oxidizer (e.g., a urea hydroxide powder (also known as a urea hydrogen peroxide or carbamide peroxide)); transporting the resulting filled package to a remote site (e.g., a hair salon) while maintaining the package to be substantially free of liquid; and optionally instructing a user to dissolve (or otherwise convert into a liquid) the oxidizer, mix it with a hair colorant or a combination thereof, for forming a hair coloring product.

In another specific aspect, the package includes an integrated measuring device adapted for metering a predetermined portion of the first predetermined amount of the granulated mixture from the package.

In yet another specific aspect, the concentrate (though possibly a gel, paste, slurry or other dispersion) is a solid state (e.g., granulated) mixture, particularly one including an oxidizer such as urea peroxide (e.g., of at least 0.05 kilogram of a mixture of urea hydroxide powder, a chelating agent, and optionally a thickening agent). By way of example, hair coloring product package may be filled so that the contents consist essentially of a granulated mixture comprising (a) urea peroxide; (b) an acrylic thickening agent in an amount sufficient for obtaining a viscosity upon dilution of about 2500 to about 150,000 cPs at about 25° C.; and (c) about 0.01 to about 5% by weight EDTA; and (d) a desiccant.

As can be seen from the above, and gleaned from the following, the present invention offers one or more advantages over existing technologies, such as (without limitation) the ability to provide a hair coloring product, and specifically a granulated oxidizer, that weighs less than a fully diluted liquid version (e.g., more than 50%, more preferably more than 65%, and still more preferably more than 80% less than the weight of a liquid oxidizer). Not only are shipping costs reduced, but less packing material is required to ship than for shipping diluted liquids. For a number of applications, lighter overall weight also makes the material more suitable for handling and storage than diluted liquids. It also provides a way to provide longer term stability of products, because liquid oxidizers can be prepared fresh closer to time of use in controlled doses.

### DESCRIPTION OF THE DRAWINGS

FIG. 1 is a view of an illustrative package for use in the present invention.

### DETAILED DESCRIPTION

As indicated, the present invention is predicated upon the discovery of improvements for methods for packaging and delivering hair coloring products, and particularly concentrated oxidizer in relatively large amounts such as for use in hair salons where multiple batches (e.g., 3, 5 or even 10 or more) of a hair coloring product can be prepared from a single package of the oxidizer. In one aspect, the invention includes steps of causing a dry and empty package to be filled to a predetermined level with a concentrated oxidizer, and more particularly a granulated oxidizer; transporting the resulting filled package while maintaining the package to be substantially free of moisture; and optionally thereafter causing a liquid to be formed that includes the granulated oxidizer alone or with a hair colorant, for forming a hair coloring product.

One unique aspect of the present invention is directed to the manner in which a concentrated oxidizer such as a granulated mixture (particularly one including an oxidizer consisting essentially of urea peroxide powder) is handled economically upon manufacture so that the oxidizer can be delivered substantially in tact at a desired concentration to a user (particularly one located at a remote location), without substantial handling concerns for users. Accordingly, it is contemplated that the concentrated oxidizer component will be handled by containing it within one or more packages, and (particularly for large quantities) a plurality of such packages will be aggregated with a common shipping holder.

Any of a number of different packages may be useful in accordance with the present invention for carrying the concentrated oxidizer component. In one preferred embodiment, the package is configured for reproducibly metering a predetermined quantity of the concentrated oxidizer. Thus one such preferred method and further includes a step of metering a predetermined quantity of the concentrated oxidizer (e.g., the granulated component) into a receptacle for receiving a liquid, a hair colorant or a combination thereof.

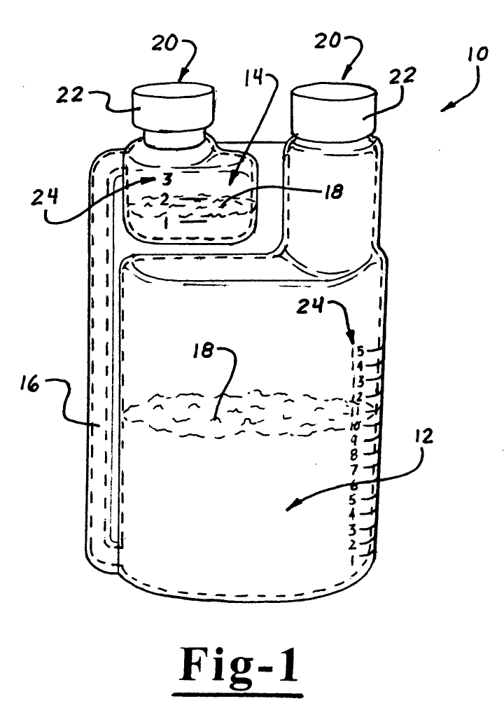

With reference to FIG. 1, an example of one such package 10 includes a squeezable dispensing reservoir portion 12 and further includes a measuring and dispensing receptacle 14 fluidly connected with the reservoir portion 12 by a duct 16, for permitting contents 18 (whether in the form of a solid, liquid, gel, paste or any combination thereof) to be squeezed from the reservoir portion 12 to the receptacle 14. Two or more of the reservoir portion 12, the receptacle 14 or the duct 16 can be integrally formed with each other. One or more openings 20 through which contents (such as granules, a diluent or otherwise) can be filled or dispensed are also included in the package 10. One or more suitable closures 22 (which optionally may be re-sealable, vented or both) may be employed as well. The closures may be adapted for covering a relatively small opening (e.g., about 1 to 10 cm² in area), or larger (e.g., more than about 12 cm², such as 20 cm²). The openings may be optionally carry a mesh or perforated shaker membrane (not shown) for controlling passage of contents therethrough. Suitable markings 24, such as fill line indicators may be applied to the reservoir portion 12, the receptacle 14 or both. Additional details about one such suitable package can be found in Canadian Patent No. 1146507, hereby incorporated by reference. In usage, the contents (e.g., granules) 18 (whether first dissolved or not) may be urged from the reservoir portion 12 through duct 16 and into the receptacle 14. For example, in one aspect, this may be preformed by closing the opening 20 to the reservoir portion 12 and maintaining the opening 20 to the receptacle 14. When the reservoir portion 12 is squeezed, the oxidizer (which may be already liquid, such as by previously filling the reservoir with water to dissolve the granules) is urged through duct 16 and into receptacle 14 until the desired amount is displaced, which may be monitored using the markings 24 associated with either or both of the reservoir portion 12 or the receptacle 14.

Of course other package configurations are possible as well, including but not limited to multiple use packages, single use packages, soft walled foil packs, blister packs, sealed trays, clamshell packaging, vials, jars, cans, bottles, cardboard boxes, bags, tubes, envelopes, or the like. Packages may be suitably adapted to be re-sealed upon opening (such as by a twist off cap, by an associated adhesive closure tab, by an insertion tab or the like).

Though other materials may be employed, the package is preferably made of a recyclable material, is refillable, or both, and thus aspects of the invention further include optional steps of recycling the package, re-filling the package or a combination thereof. By way of example, the package material may be a glass, but is more preferably a plastic, particularly a thermoplastic, and still more particularly is a thermoplastic selected from polyethylene terephthalate, high density polyethylene, low-density polyethylene, polypropylene, polystyrene, polyvinyl chloride or any combination thereof. The package material itself may be coated (e.g., with a plastic barrier film, a ceramic coating or otherwise), or otherwise surface treated. The package may include an at least partially transparent portion, an opaque portion or a combination thereof. For example, if it is desired to be able to view the contents of a closed package, the package may be equipped with a transparent window, or it may be substantially entirely transparent. It is possible also that the reservoir portion 12 is adapted for reducing exposure of contents to light (e.g., by using a colorant in the package material that acts as a filter, by applying a label, by printing an image on the package, any combination thereof, or the like) to help preserve long term stability of contents, but the receptacle 14 optionally is not so adapted (e.g., it remains sufficiently clear that a user can see the amount of contents that are dispensed into it). The package may have one or a combination of a label or instructions for use adhered to it, printed on it, embossed in it or otherwise affixed to it.

Preferably associated with each package that is adapted for repeated opening and closure will be a suitable lid, cap or other sealing device. One preferred sealing device is adapted so that it can vent accumulated gas from within sealed package to ambient, so that internal pressure build-up within the package is substantially avoided, especially after exposure to substantial amounts of moisture or upon dissolution of the concentrated oxidizer. A twist-on threaded cap, for example, might have a vent (e.g., a one-way vent) defined along an inner wall, the vent (e.g., a one-way vent) may be formed within a seal member associated with the top wall of the sealing device, a one way valve may be associated with the cap or located elsewhere in the package, or some other approach to venting may be employed. Preferably, the sealing device used in accordance with the present invention will facilitate maintaining freshness of the interior of the package and its contents, upon sealing and until opened by a user, substantially free of liquid, and particularly is free of any moisture beyond ordinary ambient amounts. One or more additional devices may be employed for effecting a seal, such as a protective seal bonded over the opening of the package, made of plastic, paper, metal foil, or a combination thereof. A fibrous mass, such as a wad of cotton, gauze or the like, may also be inserted into the package. Any cap that is employed may also include a supplemental securing device, such as a radial zip strip, a tamper-evident seal, a child-resistant cap, or any combination thereof. It may also be possible that the package omits an opening, but one is made by a user at point of use, such as by rupturing a wall. As to the latter, therefore, it is further contemplated that a cap is included with the package for use after rupture.

It is also possible that incorporated into any sealing device or elsewhere on or within the package, may be a suitable device that carries a desiccant (e.g., activated carbon, silica gel, a combination thereof or otherwise). For example, the device may include a moisture permeable membrane for allowing moisture to pass into contact with the desiccant. It may be a perforated body, such as that disclosed in U.S. Pat. No. 4,093,105, incorporated by reference. As described elsewhere herein, yet another approach is to disperse an agent among any mixture within the package that is selectively hydrophilic. For example, less than about 5% by weight (more preferably less than about 2% by weight) of fumed silica powder may be employed in a mixture with other ingredients for the concentrated oxidizer (e.g., with urea peroxide powder alone or with any other additive). Other configurations and approaches are also possible for the employment of a suitable desiccant.

As mentioned, one or more filled packages may be assembled with a common shipping holder. Such a holder preferably will meet the requirements established for transporting class 5.1 oxidizers (see 49 CFR 173, incorporated by reference), and also will be such that when containing one or more packages of concentrated oxidizer, will be regarded as a Packing Group III shipment, namely it will be regarded as a low hazard material. Any of a variety of shipping holders may be employed, such as without limitation, such as a cardboard box, a plastic or metal drum, a tote bin, a basket, a pail, a bag, an envelope, a tray, a pallet, a crate or the like. Packages may optionally be partially or fully enclosed by a protective covering (e.g., a plastic film overlayer over the entire package, over any cap or other opening portion of the package, or otherwise). Thus, a form of shipping holder also contemplates an aggregate of packages that are bundled and secured together with a plastic film, tape, strips, or the like. Further, when associated with the shipping holder, there may also be included any suitable art disclosed packing material, e.g., paper, foam plastic, beads, or the like. In addition, there may be included a suitable medium for managing spills of granulated oxidizer. For example, a tacky material may be employed for contacting and at least temporarily adhering to the granules and confining them to a particular location.

The amount of concentrated oxidizer filled into each package can vary depending upon the intended application, the size of the shipping holder, or some other reason. Typically, however, the quantities that are dispensed will be on the order of about 0.001 to about 5 kg per package, and more preferably about 0.03 to about 2 kg per package (e.g., about 0.05 to about 1 kg per package). One particularly preferred approach is to dispense into the package a salon-use quantity of concentrated oxidizer in an amount sufficient that at least three (more specifically at least five, and possibly even at least ten) separate batches of hair coloring products can be prepared from the contents of a single package, thereby reducing the number of processing steps that must be performed by a technician at a salon as compared with single use package quantities.

It should be recognized that any step of filling herein contemplates partial or complete filling. Thus, a package may be filled to its desired weight, but from a perspective of volume, less than about half of the package (or a smaller volume) is filled. It is therefore possible that any subsequent conversion of the concentrate into a liquid or other non-solid form is carried out within the package itself (e.g., by adding water to the reservoir portion), in a separate mixing receptacle or a combination thereof.

It should be appreciated that one particular aspect of the present invention also envisions filling a plurality of different packages with the employment of granulated oxidizer that is formulated to have differing concentrations of the oxidizer component (e.g., about 3% by weight oxidizer, about 6% by weight oxidizer, about 9% by weight oxidizer, about 12% by weight oxidizer or otherwise), different amounts (e.g., about 0.05 kg, about 0.1 kg, about 0.5 kg, about 1 kg, about 2 kg, about 5 kg or otherwise), having different additives (as discussed herein), or any combination thereof. Larger or smaller amounts are also possible. The present invention thus contemplates kits that contain plural packages each differing by such a characteristic. In one preferred approach, a kit may be provided that includes a plurality of packages, each being the same volume, but being filled with a different weight of oxidizer. In this approach, it is contemplated that a user will use the respective packages for mixing the powder with a liquid, such as water. As a result, the user will be able to obtain a predetermined concentration of oxidizer, based upon amount of granules originally filled. Any such kits may be assembled together with a common shipping holder for shipping in accordance with the above discussion.

Filling of the packages with the powdered peroxide can be done using art-disclosed filling techniques, such as those employed for filling containers with particulates. Filling can be performed manually or by a method or equipment that includes automated steps or components. Accordingly, it is contemplated that filling may employ steps such as repeatedly directing an amount of granules (such as those contained in a bulk storage container) into the package, while measuring (e.g., weighing) the filling until a predetermined amount of granules have been added. Alternatively, a predetermined amount of granules may be added to the package, and measured during or after the addition, followed by a step of adding or removing granules from the package to attain the desired amount. In another approach, a predetermined amount of granules is measured prior to placement into the package, adjusted as necessary to attain the desired amount, and then introduced into the package. Optionally, one or more electronic devices (e.g., controllers) will interface with a suitable measuring device (such as an electronic balance) and may optionally control a feeder to repeatedly dispense precise measurements of a material into each package.

The use of weight measurement is disclosed as one specific example, but others are possible as well, such as optical or contact sensors that monitor the height of a fill. In one aspect, it is possible that filling can be computer operated. It may also be performed so that digital fill information is obtained and stored electronically for subsequent retrieval.

Filling can be performed at the location from which the packaged granulated oxidizer is then transported to a user, or at a remote site therefrom. For example, one or more packages may be filled with granulated oxidizer at a first site where bulk dispensing quantities (e.g., in excess of about 20 kg or more) of the granulated oxidizer are maintained, and then transferred to a second site from which the filled package or packages is prepared (e.g., placed into the shipping holder, inventoried, labeled, or otherwise processed) for shipping to a customer. Though the first and second sites may be located at a common facility, they may be located also at geographically remote sites. It is possible that upon filling a plurality of packages (which each may be filled with approximately the same amount or concentration of oxidizer, or different amounts or concentrations), that the plurality of packages are assembled for transport of the plurality of packages with one or more common shipping holders to a third site that is geographically remote from the second site. Thus, typically, the third site will be a hair salon, a retail store, a warehouse or other storage or distribution facility for a salon or retail store, or some other site operated by or under the control of a customer or distributor of the product.

The shipping holder may also have included therein for shipment to a customer one or a combination of additional hair or other salon products such as, without limitation, foil (e.g., pre-cut metal foil sheets), lightener or other colorant, booster, highlighting gel, measuring device, mixing container, hair color levels swatch, a color chart, instructions (e.g., written, audio, video or a combination), shampoo, comb, hair brush, hair dryer, sculpting product, applicator, conditioner, or head caps.

The step of shipping in accordance with the present invention may make use of art-disclosed shipping methods (e.g., by rail, by waterway, by road, by air or any combination thereof). In a preferred approach shipping of packages filled with granulated oxidizer is carried out by shipping of class 5.1 oxidizers in accordance with 49 CFR 173, incorporated by reference, and particularly the packages (or shipping holders in or on which they are aggregated) will be handled as a Packing Group III shipment, namely it will be regarded as a low hazard material. It is possible that shipping is also performed in connection with an order fulfillment method, such as a method that involves offering the granulated oxidizer for sale over the internet, by advertising in a printed publication, by mail order, by phone order, or otherwise. It is also possible that the shipments are performed in accordance with a subscription service, by which a purchaser enters an arrangement for shipment of a predetermined amount of product at predetermined intervals.

During transport and storage of the concentrated oxidizer, it is preferably kept in a relatively dry and cool environment (e.g., below about 30° C.). In one aspect, the present invention envisions that the filled packages with granules herein may be storage stable at such temperature for a period of at least 6 months and more specifically at least one year. In this manner, delayed usage of the oxidizer is potentially not as wasteful as may result from delayed usage of conventional peroxide liquids.

The step of instructing can be performed in any suitable manner for helping to assure reasonably proper handling in the expected use of the granulated oxidizer. This can be accomplished in different ways, including by providing printed instructions, providing oral instructions, providing electronic instructions (e.g., via the internet), posting instructions to a user-accessible site, posting as a label on a package, instructing in an advertisement or other promotional literature, demonstrating in one or more sales demonstrations, conducting a class, any combination thereof, or the like. Once demonstrated it is still instructing even if subsequent batches omit instructions. Thus, repeated shipments in the absence of accompanying instructions would still be within a method calling for instructing users in the manner of use of granulated oxidizer herein.

The concentrated oxidizers according to the present invention may be used in methods consisting essentially of a step of bleaching hair. Thus a resulting hair coloring product need not necessarily include a colorant, but may consist essentially of the resulting bleach. More particularly, however, the powders are used in a method that includes a combination of bleaching with the oxidizer and colorant (e.g., permanent, temporary, or semi permanent) the hair. Thus, typically, the present invention is used in accordance with performing single process methods of simply applying the mixture of powder and colorant as desired throughout the hair. It may also be used in connection with double process coloring methods, where the hair is bleached to a certain level and then colorant is added to bring it to the desired level. Thus it is contemplated that the colorant that is employed (which itself may be substantially devoid of any color) is capable of interacting with oxidizer, and hair, wherein simultaneous competing reactions are occurring, namely, the bleaching of the natural pigment in the hair by the peroxide, as well as the oxidation of the coloring chemistry (e.g., art-disclosed dye precursors and couplers) for imparting color to the hair. The precursors and peroxide diffuse into the hair shaft, where color is developed. Namely, the dye precursors are oxidized by the oxidizer (also considered in the art to be a “developer”) to form intermediates that react with the couplers. The couplers, which are relatively stable to the oxidizer, react with the intermediates to form color molecules that lodge within the hair structure.

In a particularly preferred aspect of the present invention, the granulated oxidizer herein is characterized as being a substantially non-aqueous organic hydrogen peroxide complex, and even more specifically a hydrogen peroxide that is compounded with a urea (e.g., so as to be an anhydrous addition compound) and is soluble in water. For example, one specific form if the peroxide powder is a urea peroxide powder (such as represented by the formula CH₆N₂O₃). The use of powdered analogs or derivatives of urea peroxide is also contemplated herein. In addition, it is possible that other powdered oxidizing agents (e.g., including other peroxides not compounded with a urea) may be employed as well.

An example of one suitable oxidizer has a combination of at least two, more preferably three, still more preferably four, and most preferably all five of the following characteristics: (1) at least 35% by weight content of hydrogen peroxide (H₂O₂); (2) at least 16.5% by weight if active oxygen; (3) a density of about 650 g/l; (4) a pH of about 3.0 to 5.2 (for a solution ranging from 0.5-10% in demineralized water (pH 6.2)); (5) solubility in water (respectively at 20° C./40° C./60° C.) of about 500 g/l, 730 g/l, and 970 g/l.

The oxidizer may be mixed with one or more additional ingredients, such as a stabilizer, a chelating agent, a thickener, a desiccant, a fragrance, a plant extract, a nutrient, a conditioner, or a colorant (particularly one that is temporary or can be rinsed from hair for removal or that otherwise imparts no alteration to the desired hair color for an intended application). Use of a colorant for example may provide an advantage of enabling persons in the presence of a spill (whether dissolved or not) to be able to recognize the spill and distinguish it from other materials.

In one embodiment, the concentrated oxidizer is admixed to include a granulated peroxide and a chelating agent, and particularly one selected from sodium citrate, EDTA or a derivative or analog, or any combination thereof is employed. Other chelating agents may be employed as well, such as a cyclic oligosaccharide (e.g., cyclodextrin). The amount of chelating agent may range from about 0.01 to about 5% by weight, and more specifically is about 1 to about 3% by weight. In another embodiment, the concentrated oxidizer is admixed to include a granulated peroxide and a thickening agent (e.g., in an amount sufficient so that the viscosity upon dilution with a liquid such as water will be about 2500 to about 150,000, and more specifically about 4000 to about 10,000 cPs at about 25° C.). In still another embodiment, the concentrated oxidizer is admixed to include a combination of a chelating agent and a thickening agent. Other additives may also be included as discussed elsewhere herein.

Examples of suitable thickeners include, without limitation, an inorganic or organic powder (such as a powdered acrylic polymer such as available under the trade designation of Carbopol® (e.g., Carbopol® Ultrez 10). Another additive may be added for neutralizing the thickener (e.g., sodium hydroxide in saline solution), for inducing gelling or both.

Examples of other oxidizers that may be employed in the present invention include any of a number of non-aqueous peroxides, for example, as disclosed in U.S. Pat. No. 5,770,739 (incorporated by reference)(e.g., without limitation, poly(vinyl alcohol)-hydrogen peroxide complex, a poly(vinyl methyl ether)-hydrogen peroxide complex, a poly(vinyl methyl ketone)-hydrogen peroxide complex, a poly(acrylic acid)-hydrogen peroxide complex, a poly(vinyl acetate) hydrogen peroxide complex, a cellulose acetate hydrogen peroxide complex, a sodium alginate hydrogen peroxide complex, a cellulose sulfate, sodium salt, hydrogen peroxide complex, a glycine-hydrogen peroxide complex, poly(4-vinylpyridine) hydrogen peroxide complex, a histamine hydrogen peroxide complex, a propionamide hydrogen peroxide complex, a 1,3-dimethylurea hydrogen peroxide complex, a biuret hydrogen peroxide complex, a polyacrylamide hydrogen peroxide complex, a nylon 6 hydrogen peroxide complex, a nylon 6,6 film hydrogen peroxide complex, a polyetherpolyurethane hydrogen peroxide complex, and a rubidium carbonate hydrogen peroxide complex). Granules of inorganic peroxides are also contemplated within the scope of the oxidizers of the invention. Though peroxides are disclosed as one preferred approach, other granulated agents may be used for generating free oxygen such as, without limitation, those including persulfates, perborates, percarbonates, or the like. See, e.g., U.S. Pat. Nos. 5,575,989; 5,769,901; 6,302,920, all of which are incorporated by reference herein.

Yet another aspect of the present invention pertains to methods for preparing an oxidizer, and specifically urea peroxide. Although other techniques for its production are possible, an example of one approach includes a step of recrystallizing a mixture of starting materials such as aqueous peroxide, e.g., (hydrogen peroxide (H₂O₂)) and dry urea-based crystals (e.g., CO(NH₂)₂). Without intending to be bound by theory, it is believed that the following chemical equation will apply:
H₂O₂(aq)+CO(NH₂)₂→H₂O₂.CO(NH₂)₂

Any suitable concentration of peroxide solution (e.g., aqueous hydrogen peroxide) can be used in this recrystallization synthesis; however, one approach contemplates starting with an aqueous solution of about 20 to about 50 vol. %, more preferably about 25 to about 45 vol. %, and still more preferably about 33% to about 38% (e.g., about 35 vol. %) hydrogen peroxide.

While suitable urea may contain impurities and water, one specific recrystallization according to the present invention employs a dry (e.g., crystalline) urea material, particularly one that is substantially free of water and impurities. In one preferred embodiment, the dry urea material is 95% pure. In a more preferred embodiment, it is employed in a purity of between about 95% to about 99.99%; preferably a purity of between about 97% to about 99.9%; and most preferably, at least about 98% purity (e.g., 99% dry prilled urea).

In a clean reaction vessel, and preferably under a vented fume hood, the aqueous hydrogen peroxide and the dry urea are mixed (e.g., at a suitable concentration, such as about 1:3 to about 3:1, or more specifically about 1.3:1 to about 1:1.3 (and even more specifically about 1:1) molar ratio), such as by stirring. Other concentrations may be employed as well.

The mixture can be heated or cooled to temperatures between about 15° C. and about 60° C.; however, room temperature is preferred. Once substantially all of the urea crystals dissolve in the aqueous hydrogen peroxide, the solution is cooled (e.g., by air cooling, a quench or a combination thereof) at one or more rates (e.g., ranging from about 0.1 to about 10° C. per minute, and more specifically about 1 to about 5° C. per minute), to a temperature below about 10° C. (e.g., about 4° C.). During this cooling process, urea peroxide will crystallize and thus form granules in accordance with the present invention. The granules can then be isolated and dried using any suitable technique, and most preferably, by vacuum filtration. Optionally, resulting peroxide can be tested and steps repeated to assure the desired concentration level.

It should be recognized that it may be possible as part of the above disclosed exemplary process that a step of mixing additives may be employed prior to, simultaneously with or after crystallization. For example, one or both of the chelating agent or the thickener (or some other additive) may be mixed with the starting materials, mixed into the solution prior to crystallization, added to the resulting mixture of granules or any combination thereof.

Granules of the present invention, whether resulting from the above method or another, will typically be on the order of less than about 2 mm average diameter, and more preferably less than about 1 mm average diameter (e.g., about 0.5 mm average diameter or smaller). Typically, the dispersity of the granules by average diameter that are package according to the invention herein will be substantially mono-disperse, although poly-disperse mixtures are also possible (e.g., with at least two groups of different average particle diameters, each comprising between about 20 and 40 wt. % of the total grains in the mixture). Desired grain size may also be controlled by including a suitable milling step.

The granules of the present invention also contemplate that a solid or liquid oxidizer (such as one specifically including urea peroxide) may be provided in an encapsulated form, as part of an inclusion complex (e.g., a bead, a rod or the like, formed with a chelating agent such as a cyclic oligosaccharide (e.g., cyclodextrin)), or both. Granules may themselves be surface treated with one or more other agents for altering their performance characteristics. For example, granules may be treated with a surfactant for modifying the surface properties of the granules, such as for preventing clumping of the granules. Granules may likewise be treated with a hydrophobic agent for assisting the granules resist moisture. On this point, it may be possible to form an inclusion complex with the treated granule (e.g., as described above with cyclodextrin; see also, “Del Valle, “Cyclodextrins and Their Uses: A Review,” Process Biochemistry 39 (2004) 1033-1046, hereby incorporated by reference). It is also possible that a plurality of granules is compacted together into a tablet form (which optionally may be ground before dissolution). Thus granules herein contemplate particles that have a core and a coating, as well as particles without a coating. Granules may also be employed that are converted to a liquid by application of an external stimulus such as pressure, heat, radiation, or any combination thereof instead of or in addition to being dissolved in a liquid.

It is contemplated that as to any mixture that is filled into a package according to the steps herein, the oxidizer will be present in an amount greater than about 45% by weight of the mixture, more preferably greater than about 65% by weight of the mixture, and still more preferably greater than about 85% by weight of the mixture. By way of one specific example, without limitation, a hair coloring product package may contain a mixture comprising (a) greater than about 50% by weight (more preferably greater than about 65% by weight of the mixture, and still more preferably greater than about 85% by weight of the mixture) of urea peroxide; (b) a thickening agent (e.g., an acrylic thickening agent) in an amount sufficient for obtaining a viscosity upon dilution of the mixture water of about 2500 to about 150,000 cPs at about 25° C.; and (c) about 0.01 to about 5% by weight of a chelating agent (e.g., EDTA); and optionally, (d) a desiccant.

Under any of such approaches, it is contemplated that other steps that do not materially affect coloring of hair may also be performed, such as without limitation, cleansing the hair, conditioning the hair, reconstructing damaged hair, treating dandruff, nourishing the hair, curling the hair, clarifying the hair, straightening the hair, cutting the hair, styling the hair, glazing the hair or any combination thereof. Further, hair that is treated need not be the entire body of hair, but may be selective portions (e.g., for achieving highlights, lowlights, or otherwise).

In yet another aspect of the present invention, it is contemplated that a receptacle is provided within which the granules are dissolved prior to application to hair. In one embodiment, the receptacle is part of a cap for the package that holds the granules. In another embodiment, the receptacle is provided separately from the package (e.g., a measuring cup, spoon or the like). In yet another embodiment, such as for single use only applications, the receptacle is integral with the package itself. For example, the receptacle may include at least two compartments, divided by a partition, the latter of which may be rupturable for exposing granules contained within one of the compartments with solvent (e.g., water) that is added to another of the compartments. Additional embodiments are also possible, such as packaging granules in a water soluble packet and then contacting the packet with water for activating the granules, in accordance with U.S. Pat. No. 5,116,388, incorporated by reference.

Based upon the above, accordingly, it will be appreciated that the present invention also addresses methods dispensing of granules at point of use by a user, treating hair with dispensed granules or both. In one respect, granules are dispensed by using the package containing them for dispensing a metered portion of total granules filled in the package, using a measuring device that is part of the package itself or the cap that seals it or both. In another respect, granules can be dispensed into a receptacle using a separate measuring device.

The methods herein contemplate not only steps such as filling, transporting, storing, or other handling steps, but also contemplate steps of offering the products for sale and consummating sales of products. The latter steps may be performed in person, by print media, by radio, by television, by internet, or any combination thereof. Thus steps including consummating a sales transaction over the internet, by telephone, or by mail order are also within the invention.

By way of one illustrative example for preparing a peroxide granule in accordance with the present invention, a 100 mL beaker equipped with a magnetic stirring bar and a thermometer is charged with about 25.5 g (0.22 mol) of about 30% aqueous hydrogen peroxide solution. Stirring is begun and about 9.01 g (0.15 mol) of urea added. The beaker then is heated slowly to a temperature of about 60° C. Dissolution of the urea is observed in the temperature range of about 25-30° C. Once the temperature reaches about 60° C. the solution is held at that temperature for a few additional minutes. The resultant solution is removed from the heat source, and left to stand to slowly cool to room temperature. Upon standing in the open air in a fume hood, crystals form. If crystallization is not observed once the beaker has come to room temperature, gentle swirling of the solution can be employed to induce crystallization. The resultant crystals are isolated by vacuum filtration, then transferred to a desiccator containing calcium chloride and stored desiccated in a refrigerator (about 4° C.). Yield typically is in the range of 4 g (28%); higher chemical yields are possible by cooling the initial solution in an ice water bath prior to vacuum filtration, by similar cooling of the filtrate or both. Resulting crystals exhibit a melting point of about 84-93, and more specifically 86-88° C. (dec) with prior softening.

By way of further example to illustrate one specific concentration differentiation approach, without limitation, approximately 177 grams of a urea peroxide powder are added to a package as in FIG. 1, with instructions to add to the package approximately one-half liter of treated water (e.g., bottled water) or tap water to make a 40 Volume composition of a hair color developer, which (like the other examples herein) can then be dispensed in predetermined amounts smaller than the package total resulting volume of developer. Similarly, approximately 88.5 grams of urea peroxide powder are added to a package, with instructions to add approximately one-half liter of water to make a 30 Volume composition of a hair color developer. Likewise, approximately 177 grams of urea peroxide powder are added to a package, with instructions to add approximately one liter of water to make a 20 Volume composition of a hair color developer. Moreover, approximately 88.5 grams of urea peroxide powder are added to a package, with instructions to add approximately one liter of water to make a 10 Volume composition of a hair color developer. The resulting 10 Volume, 20 Volume, 30 Volume and 40 Volume products can be offered, shipped or both, either individually or in combinations of two or more of the same or different Volume types.

From the example of the immediate preceding paragraph, it is seen that a plurality of different packages are provided, with each having a predetermined amount of granules for achieving a predetermined concentration of oxidizer upon dissolution with a predetermined amount of a liquid (e.g., water). As gleaned from the teachings herein, concentrations of oxidizer can be varied by any of a number of different approaches. For example, a user can dispense, from a single package, a plurality of differing amounts of granules into different respective receptacles, each of which is dissolve with the same predetermined volume of liquid (e.g., water). A user can dispense, from a single package, the same amounts of granules into different respective receptacles, each of which is dissolved with a different predetermined volume of liquid (e.g., water). Of course, other variations are also possible.

For purposes herein, the determination of hydrogen peroxide concentrations in an aqueous solution may be performed using any suitable approach. An example of one particular approach is set forth at http://www.degussa-huels.co.nz/downloads/Application%20-%20Analytical%20Method.pdf#search=‘iso%207157’) (also referencing ISO 7157), incorporated by reference. According to that approach, the reaction of potassium permanganate with hydrogen peroxide in acidic medium is monitored according to the following equation:
2 KMnO₄+5H₂O₂+4H₂SO₄->2 KHSO₄+2 MnSO₄+8H₂O+5 O₂.
The procedure may be carried out manually or, when using automated modern titration equipment. Approximately 50 ml 5N sulphuric acid (H₂SO₄, 2.5 mol/l) is added in a flask along with an exactly weighed sample (e.g., by using an analytical balance accurate to four decimal digits)). A syringe, rinsed thoroughly before use, is used to add the H₂O₂ sample, with the amounts preferably weighed in the filled syringe before and after adding a few drops to the flask. By way of example, the following sample weights may be used: (a) H₂O₂ 30-35%: 0.3-0.4 g sample weight; (b) H₂O₂ 50%: 0.2-0.3 g sample weight; and H₂O₂ 70% and above: 0.1-0.2 g sample weight. For all procedures, deionised or distilled water is used. Under vigorous mixing, the sample is titrated with a regularly standardized 0.25N potassium permanganate solution (0.05 mol/l). If visible indicators are used, then titration is performed until a pale pink coloring persists. If using automatic titration equipment, the end point can be determined according to the type of instrumentation used (e.g., potentiometrically via a redox electrode). In case a different concentration of KMnO₄ is used (e.g., 0.1N KMnO₄), the numbers in the calculation formula can be adjusted accordingly. Hydrogen peroxide in the solution is calculated as follows: % H₂O₂=ml of consumed KMnO₄ solution (0.05 mol/l)×0.425175 sample weight [g]. This provides the concentration of hydrogen peroxide in solution expressed in weight percent (g H₂O₂/100 g solution).

The specific embodiments herein, though disclosed in the context of granulated oxidizer components, are not intended to limit the invention to coverage of other forms of the oxidizers, especially if at least one of the advantages of the invention are realized, such as the ability to more economically or easily handle, package, or transport the oxidizers, as compared with conventionally filled packages of full-strength liquids. As indicated, other such forms may include, for example, pastes, gels, slurries, or other dispersion.

In addition, though described herein with respect to hair care products, the methods may also find other suitable applications. For example, hair care applications may be substituted with dental care applications for the above embodiments. Thus, it is contemplated that methods herein may envision the packaging and transport of oxidizers, and especially granulated oxidizers such as urea peroxide, for dispensing or use in a dental care facility, a health care facility, or a pharmacy. In this manner, bulk quantities of oxidizers for teeth bleaching applications may be handled according to steps described above for employment with the hair care products.

It will be further appreciated that functions or structures of a plurality of components or steps may be combined into a single component or step, or the functions or structures of one step or component may be split among plural steps or components. Alternatively, functions performed by one of the components might be split among or performed by other components (e.g., splitting the package into a physically separate reservoir and measuring receptacle). The present invention contemplates all of these combinations. Unless stated otherwise, dimensions, geometries, amounts and concentrations of the various features depicted herein are not intended to be restrictive of the invention, and others are possible. In addition, while a feature of the present invention may have been described in the context of only one of the illustrated embodiments, such feature may be combined with one or more other features of other embodiments, for any given application. It will also be appreciated from the above that the fabrication of the unique structures herein and the operation thereof also constitute methods in accordance with the present invention. The present invention also encompasses intermediate and end products resulting from the practice of the methods herein. The use of “comprising” or “including” also contemplates embodiments that “consist essentially of” or “consist of” the recited feature.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

1. A method for delivering hair coloring products, comprising the steps of:
a) causing an empty package be filled to a predetermined level with a solid state oxidizer in an amount sufficient so that at least three separate batches of hair coloring products can be prepared from the contents of the package;
b) transporting the resulting filled package to a remote site while maintaining the package to be substantially free of liquid and the oxidizer in its solid state; and
c) instructing a user to at least partially dissolve the solid state oxidizer for forming a hair coloring product.

2. The method of claim 1 wherein the package includes a recyclable molded thermoplastic receptacle portion for housing the solid state oxidizer.

3. The method of claim 2, wherein the package includes a vented cap for helping to prevent gas pressure buildup within a closed package.

4. The method of claim 3, wherein the package is configured for reproducibly metering a predetermined quantity of the oxidizer into a receptacle for receiving water, a liquid hair colorant or a combination thereof.

5. The method of claim 1, further comprising the step of metering a predetermined quantity of the oxidizer into a receptacle for receiving water, a liquid hair colorant or a combination thereof.

6. The method of claim 3, wherein the causing step (a) is performed a plurality of times for filling a plurality of packages, and further comprising a step of assembling the resulting plurality of packages for transport of the plurality of packages via a common shipping holder to the remote site.

7. The method of claim 1, further comprising a step of adding to the package an additive mixture that includes a chelating agent and at least one additive selected from a stabilizer, a dessicant, a thickening agent, a fragrance, a plant extract, a nutrient, a conditioner, a colorant or any combination thereof.

8. The method of claim 7, wherein the chelating agent includes up to about 3% by weight of EDTA, and the additive includes a thickening agent in an amount sufficient to realize a viscosity upon dilution with a liquid such as water of about 2500 to about 150,000 at about 25° C.

9. The method of claim 4, wherein the package includes a squeezable dispensing reservoir portion and further includes an integral measuring and dispensing receptacle fluidly connected with the reservoir portion.

10. The method of claim 1, wherein the remote site is a hair salon.

11. A method for delivering an oxidizer to a remote site, comprising the steps of:
a) filling a re-sealable and refillable, molded thermoplastic package with a mixture, including an oxidizer consisting essentially of urea peroxide powder;
b) venting the package; and
c) instructing a user to convert the mixture into a liquid.

12. The method of claim 11, further comprising a step of transporting the resulting filled package to the remote site.

13. The method of claim 12 wherein the step of transporting includes transferring the package to the remote site as a class 5.1 oxidizer material per 49 CFR 173, as a Packing Group III shipment.

14. The method of claim 11, wherein the package includes a squeezable dispensing reservoir portion and further includes an integral measuring and dispensing receptacle fluidly connected with the reservoir portion.

15. A re-sealable molded and vented package filled with urea peroxide powder resulting from the method of claim 11.

16. A method for delivering hair coloring products, comprising the steps of:
a) filling a plurality of vented packages of at least about 0.5 liters volume with at least 0.05 kg of granules of an oxidizer that consists essentially of urea peroxide, substantially in the absence of moisture;
b) assembling the plurality of packages for transport of the plurality of packages via a common shipping holder to a hair salon where it will be mixed with water and a hair colorant; and
c) causing the filled packages to be transferred to the hair salon, where the granules will be mixed with water, as a class 5.1 oxidizer material per 49 CFR 173, as a Packing Group III shipment.

17. The method of claim 16 wherein the contents of the plurality of the packages differ in respect to amount of urea peroxide powder, concentration of urea peroxide powder, or both and wherein the contents further include a chelating agent and a thickening agent.

18. The method of claim 17 wherein the package includes a squeezable dispensing reservoir portion and further includes an integral measuring and dispensing receptacle fluidly connected with the reservoir portion by a duct.

19. The method of claim 16, further comprising a step of causing to be shipped with the filled packages at least one additional product selected from foil, colorant, booster, highlighting gel, measuring device, mixing container, hair color levels swatch, a color chart, instructions, shampoo, comb, hair brush, hair dryer, sculpting product, applicator, conditioner, head cap, or any combination thereof.

20. A hair salon product shipment resulting from the method of claim 16.

21. A solid state hair coloring product adapted for dilution by a user at a point of use, comprising a powdered peroxide; a thickening agent; a chelating agent.

22. The hair coloring product of claim 21, wherein the powdered peroxide is urea peroxide.

23. The hair coloring product of claim 22, wherein the thickening agent is a polymeric thickening agent in an amount sufficient for obtaining a viscosity upon dilution of the granulated mixture with water of about 2500 to about 150,000 cPs at about 25° C.; and further comprising about 0.01 to about 5% by weight EDTA.

24. The hair coloring product of claim 23, wherein the thickening agent is an acrylic thickening agent.

25. The hair coloring product of claim 23, further comprising a desiccant.

26. A granulated hair coloring adapted for dilution by a user at a point of use, consisting essentially of a mixture of (a) urea peroxide; (b) an acrylic thickening agent in an amount sufficient for obtaining a viscosity upon dilution of the granulated mixture with water of about 2500 to about 150,000 cPs at about 25° C.; and (c) about 0.01 to about 5% by weight EDTA.

27. A hair coloring product package the contents of which consist essentially of a mixture comprising (a) greater than about 50% by weight of urea peroxide; (b) an acrylic thickening agent in an amount sufficient for obtaining a viscosity upon dilution of the mixture with water of about 2500 to about 150,000 cPs at about 25° C.; and (c) about 0.01 to about 5% by weight EDTA; and (d) a desiccant.

28. The hair coloring product package of claim 27 wherein the mixture is in a solid state.

29. The hair coloring product package of claim 28, wherein the urea peroxide is in a particulated state and is present in an amount greater than about 85% by weight.
